(19) United States
(12) Patent Application Publication
(10) Pub. No.: US 2023/0240527 A1
(43) Pub. Date: Aug. 3, 2023

(54) AN EYE EXERCISER AND A METHOD FOR RELIEVING EYE STRAIN

(71) Applicants: Uday Purushottam CHAUDHARI, Pune (IN); Mamta Uday CHAUDHARI, Pune (IN)

(72) Inventors: Uday Purushottam CHAUDHARI, Pune (IN); Mamta Uday CHAUDHARI, Pune (IN)

(73) Assignee: Mistica Innovations Private Limited, Pune (IN)

(21) Appl. No.: 18/000,292

(22) PCT Filed: May 26, 2021
(86) PCT No.: PCT/IN2021/050511

§ 371 Date: Nov. 30, 2022

(30) Foreign Application Priority Data
Jun. 2, 2020 (IN) 202021023077

(51) Int. Cl. A61B 3/113

(52) U.S. Cl. CPC A61B 3/113; A63B 2220/20

(57) ABSTRACT

Eye exerciser (100) provides better exercising efficiency because of achieving more focus, consistency and preciseness and provision of controlled movement and selected movement. The eye exerciser (100) includes an object (10) and a movement mechanism (20). The movement mechanism (20) is configurable to direct controlled movement to the object (10) such that the object (10) attains at least one selected movement. The selected movement of object (10) is selected from the group consisting of: rotational, revolutionary, simultaneous rotational and revolutionary, vertical, horizontal and oscillation movements. The best method is providing simultaneous rotational movement and revolutionary movement of object (10) which provides left to right, right to left, up to down, down to up, clockwise, counter clockwise, near and far movements of the eye or eyes (12) of the user (11) and thus relieve eye strain.

### FIELD

The present disclosure relates to an eye exerciser, wherein an object is moved in selected directions and a user's eye(s) follow the movement of the object and the movement of eye(s) results in eye exercise. Particularly, the present disclosure relates to a mechanical eye exerciser that has an object and the movements of the object are controlled to move in selected directions.

### BACKGROUND

Eye strain reducing devices are known in the prior art, for example from patent documents U.S. Pat. Nos. 6,139,149, 7,255,439, 2,224,776 and 1,464,075, which when observed by user's eyes relieves eye strain. The device to reduce eye strain of the patent document U.S. Pat. No. 6,139,149 discloses visual elements freely suspended and moves by movement of the surrounding air and hence there is no controlled movement of visual elements. Uncontrolled movements may result in lack of focus, consistency, and preciseness. The eye diversion assembly and method of U.S. Pat. No. 7,255,439 though has controlled movement but has multiple panels rotating that may disturb the user. Also, the rotation of panels is a simple rotation in one plane and hence does not provide adequate eye exercise. The eye exercising device of the patent document U.S. Pat. No. 2,224,776 discloses rolling of object in a trackway by tilting the trackway by manipulating a handle. The object is observed from a chin rest provided on the device which causes inconvenience to adjust height between the user and the device. The eye-exercising device of U.S. Pat. No. 1,464,075 is a hand-operated device that swings object in an arc. Hand-operation may not remain stable during operation and therefore disturb the eye concentration on the object.

The known eye strain reducing devices, however, does not provide a controlled movement of object in one or more plane(s) which increases exercising efficiency because of the movement preciseness and consistency. Further, the known devices may require operation with human touch that may result in less focus for performing multiple operations.

### Objects

Some of the objects of the arrangement of the present disclosure are aimed to ameliorate one or more problems of the prior art or to at least provide a useful alternative and are listed herein below.

An object of the present disclosure is to provide an eye exerciser that has controlled movement of object any without manual intervention resulting in increased focus.

Another object of the present disclosure is to provide an eye exerciser that enable movement of a user's eyes in a single and multiple planes.

Still another object of the present disclosure is to provide an eye exerciser that is portable and easy to operate.

Yet another object of the present disclosure is to provide a method for eye exercising by an eye exerciser that provides desired eye rotation because of controlled movement of object.

Still another object of the present disclosure is to provide a method for eye exercising and relieving eye strain that increases focus and concentration of the user.

Other objects and advantages of the present disclosure will be more apparent from the following description when read in conjunction with the accompanying figures, which are not intended to limit the scope of the present disclosure.

### SUMMARY

The eye exerciser according to the invention in comparison has the advantages to provide better exercising efficiency by way of achieving more focus, consistency, and preciseness because of provision of controlled movement and selected movement. The eye exerciser includes an object and a movement mechanism. The movement mechanism is configurable to direct controlled movement to the object such that the object attains at least one selected movement. The selected movement of object is selected from the group consisting of: a rotational movement, a revolutionary movement, a simultaneous rotational movement and a revolutionary movement, a vertical movement, a horizontal movement and an oscillating movement. The rotating movement is rotation of the object around the object's rotational axis and revolutionary movement is revolution of the object around the defined-axis of the motion mechanism. In an operative configuration, the movement mechanism configured to provide the selected and controlled movements to the object and a user moves an eye or eyes following the selected and controlled movements of the object thus rendering the eye or the eyes exercise.

In one embodiment, the movement mechanism includes a base arrangement, a first rotating arrangement, an extending rod, a second rotating arrangement and a suspended rod. The base arrangement is positionable on a horizontal surface or fixable on a vertical surface. The first rotating arrangement is supported by the base arrangement. The extending rod is connected to the first rotating arrangement and extends from the base arrangement. The extending rod is controllably rotated by the first rotating arrangement. The second rotating arrangement is disposed at the extended edge of the extending rod. The suspended rod is in connection with the second rotating arrangement and suspends therefrom. The suspended rod at the free end is provided with the object. In an operating configuration, the first rotating arrangement rotates the suspended rod such that the object attains revolutionary movement around the extending rod and the second rotating arrangement rotates the suspended rod and the object to attain rotating movement. The simultaneous revolutionary movement and the rotating movement render the eye or the eyes (12) exercise.

In one embodiment, the movement mechanism includes a track which is replaceable with the suspended rod and is to be connected with the second rotating arrangement. The slider arrangement is movable on the track. The slider arrangement is connected with the object (10). The track is connected with the second rotating arrangement in at least one of: a vertical configuration to achieve the vertical movement and a horizontal configuration to achieve the horizontal movement.

A method for performing a user's eye or eyes exercise. The method includes:
providing an eye exerciser having:
   an object; and
   a movement mechanism configurable to direct controlled movement to the object such that the object attain at least one selected movement,
actuating said movement mechanism to attain the selected movement of the object and the controlled movement of the object, the selected movement are one of:
   a simultaneous rotating movement and revolutionary movement of said object;
   a rotating movement;
   a revolutionary movement;
   a vertical movement;
   a horizontal movement; and
   an oscillating movement;
wherein, the rotating movement is rotation of the object around the object's rotational axis (X) and revolutionary movement is revolution of said object around the defined-axis (Y) of the motion mechanism; and
following the controlled movement and the selected movement of the object (10) by the eye or eyes (12) of the user (11) who is at a distance from the object (10), render eye or eyes exercise.

In one embodiment, the method includes performing simultaneous revolutionary movement and rotational movement of the object by:
actuating a first rotating arrangement such that the first rotating arrangement rotates an extending rod extending from a base arrangement, said base arrangement supports the first rotating arrangement; and
actuating a second rotating arrangement such that the second rotating arrangement rotates a suspended rod. The second rotating arrangement is disposed at the extended edge of the extending rod. Rotation of the suspended rod provides rotational movement to the object connected at the free end of the suspended rod, wherein, the rotation of the extending rod results in revolutionary movement of the suspended rod and thereby the object around the defined-axis (X) of the extending rod,
following the simultaneous revolutionary movement and the rotating movement renders the eye or the eyes exercise.

In additional embodiment, the method includes performing the step of the vertical movement and the horizontal movement of the object by:
dismantling the suspended rod from the second rotating arrangement;
assembling a track on the second rotating arrangement selectively in a horizontal configuration and a vertical configuration;
mounting a slider arrangement on the track. The slider arrangement is connected with the object; and
actuating the slider arrangement such that a slider moves on the track,
wherein, the movement of the slider is dependent on the position of the track and following the movement of the slider render eye or eyes exercise.

### BRIEF DESCRIPTION OF ACCOMPANYING DRAWINGS

The present disclosure will now be described with the help of the accompanying drawings, in which:

FIG. 1 illustrates a perspective side view of an eye exerciser which is mounted on a horizontal surface and has an object which is observed by a user, in accordance with one embodiment of the present disclosure;

FIG. 2 illustrates a perspective front view of the eye exerciser of the FIG. 1;

FIG. 3 illustrates an exploded view of the eye exerciser of the FIG. 1;

FIG. 4 illustrates a side view the eye exerciser of the FIG. 1, wherein the eye exerciser is fixed on a vertical surface;

FIG. 5 illustrates a side perspective view of the eye exerciser that is provided with simultaneous rotation and revolution of the object and the user ball movement in the spherical movement;

FIG. 6 illustrates a perspective view of the eye exerciser of FIG. 1, wherein the object in a first down position;

FIG. 7 illustrates a perspective view of the eye exerciser of FIG. 1, wherein the object in a second left position;

FIG. 8 illustrates a perspective view of the eye exerciser of FIG. 1, wherein the object in a third top position;

FIG. 9 illustrates a perspective view of the eye exerciser of FIG. 1, wherein the object in a fourth right position;

FIG. 10 illustrates a perspective view of the eye exerciser of FIG. 1, wherein the object in a fifth back position;

FIG. 11 illustrates a perspective view of the eye exerciser of FIG. 1, wherein the object in a sixth top-left position;

FIG. 12 illustrates a perspective view of the eye exerciser of FIG. 1, wherein the object in a seventh top-right position;

FIG. 13 illustrates a perspective view of the eye exerciser of FIG. 1, wherein the object in an eight bottom-right position;

FIG. 14 illustrates a perspective view of a track having a curved surface and a slider arrangement for providing horizontal movement of the object;

FIG. 15 illustrates an exploded view of a track having a curved surface and a slider arrangement for providing horizontal movement of the object;

FIG. 16 illustrates a perspective view of a track having a flat surface and a slider arrangement for providing horizontal movement of the object;

FIG. 17 illustrates an exploded view of a track having a flat surface and a slider arrangement for providing horizontal movement of the object;

FIG. 18 illustrates a perspective view of a track having a curved surface and a slider arrangement for providing vertical movement of the object;

FIG. 19 illustrates an exploded view of a track having a curved surface and a slider arrangement for providing vertical movement of the object;

FIG. 20 illustrates a perspective view of a track having a flat surface and a slider arrangement for providing vertical movement of the object; and

FIG. 21 illustrates an exploded view of a track having a flat surface and a slider arrangement for providing vertical movement of the object.

### DETAILED DESCRIPTION

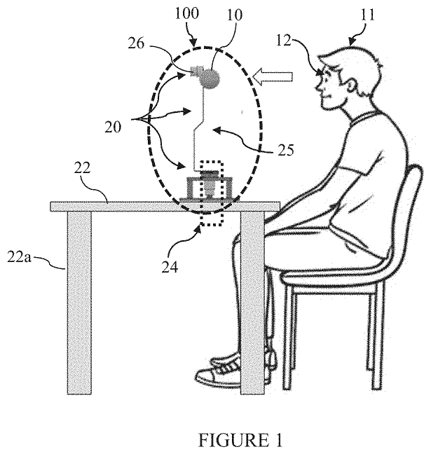

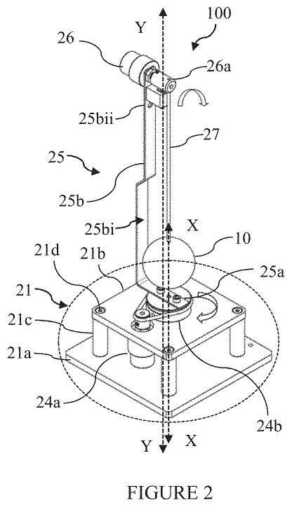

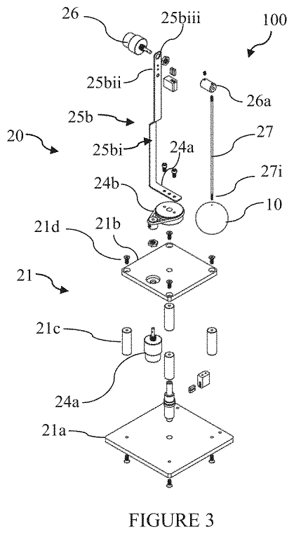

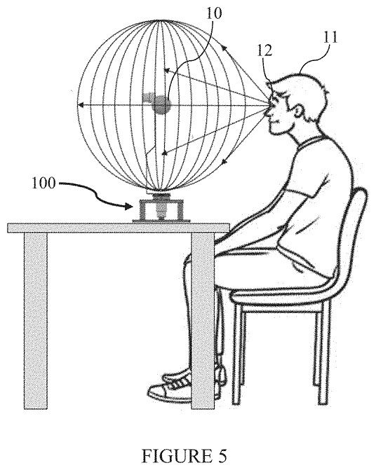

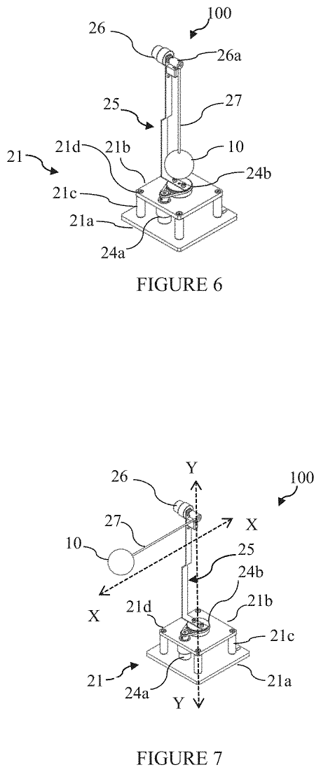

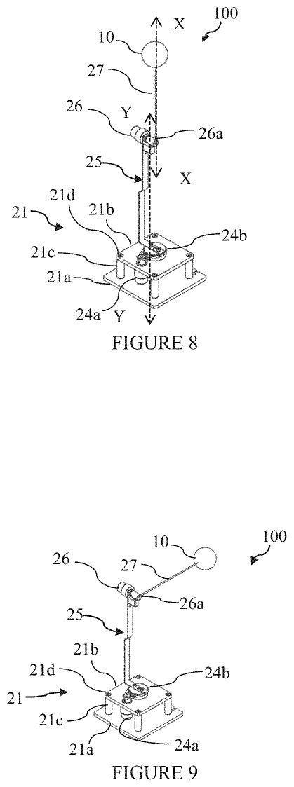

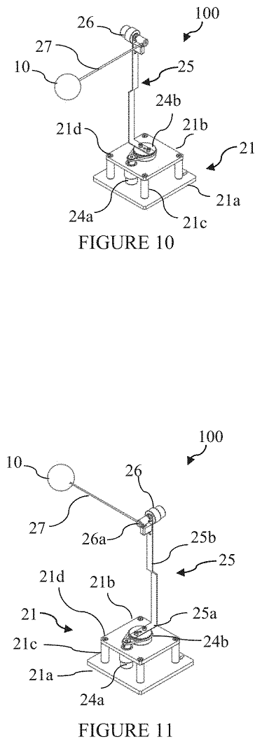

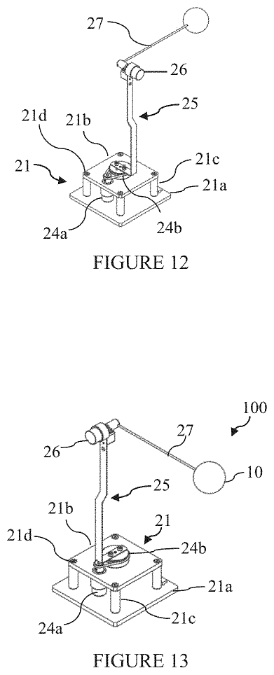

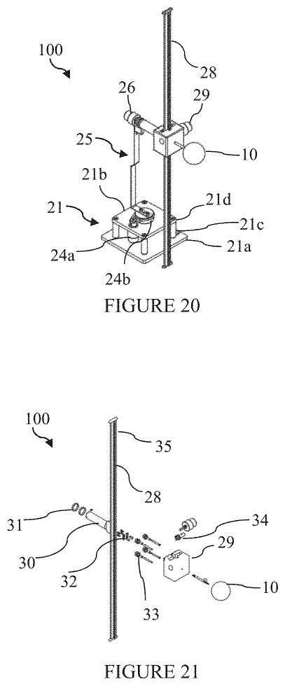

Referring now to the drawings, FIGS. 1 to 21, where the present invention is generally referred to with numeral (100), it can be observed that an eye exerciser, in accordance with an embodiment, is provided for a user (11) to perform eye exercise and the eye exerciser (100) includes an object (10) and a movement mechanism (20). The user (11) can have either one eye or both the eyes (12) that observe the eye exerciser to perform exercise for the eye or the eyes (12).

The object (10) can be of any shape, size, configuration, color, material and texture. In typical illustration of Figures, the object is spherical shaped. The object (10) is desired to be of the size which is visible by a human/a user (11) by naked eyes (12). The object (10) can be three-dimensioned (as illustrated in Figures) or two-dimensioned like an eye-catcher drawing having a big spot.

The movement mechanism (20) provides different movements to the object (10). The different movements of the object (10) can be selected by the requirement of the user (11). The different movements which the user can select are a rotational movement, a revolutionary movement, a simultaneous rotational movement, a vertical movement, a horizontal movement and an oscillating movement. After selection of the movement, the user (11) can configure the movement mechanism (20) to achieve the selected movement of the object (11). Herein, the rotating movement is rotation of the object (11) around the object's rotational axis (X) and the revolutionary movement is revolution of the object (11) around the defined-axis (Y) of the motion mechanism (20).

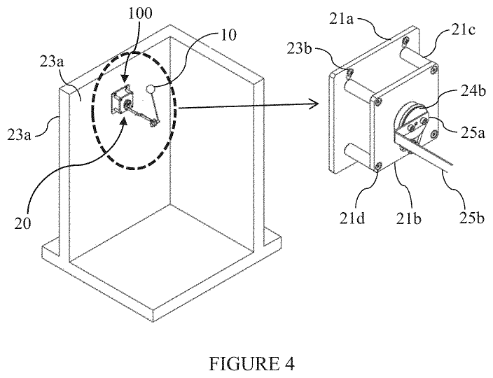

In accordance with one embodiment, the movement mechanism (20) is configured with the use of a base arrangement (21), a first rotating arrangement (24), an extending rod (25), a second rotating arrangement (26) and a suspended rod (27). The base arrangement (21) can be positioned on a horizontal surface (22)(as illustrated in FIG. 1) or can be fixable on a vertical surface (23)(as illustrated in FIG. 4). In one embodiment, the base arrangement (21) has a lower support (21a) and an upper support (21b). The lower support (21a) can be supported on a horizontal surface (22) such as a table (22a) or alternatively can be fixed on a vertical surface (23) such as a wall (23a) by use of fasteners (23b). The lower support (21a) and the upper support (21b) are space apart by positioning supporting elements (21c) therebetween. The supporting elements (21c) are fixedly fitted with the lower support (21a) and the upper support (21b) use of fasteners (21d). The first rotating arrangement (24) includes a rotating motor (24a) and a power transmission mechanism. The rotating motor (24a) is disposed in the space from between the lower support (21a) and the upper support (21b). As illustrated, the power transmission mechanism is a belt-drive transmission mechanism (24b). However, the power transmission mechanism of the present disclosure is not limited to the use of the belt drive transmission mechanism (24b) and other power transmission mechanisms such as gear transmissions, chain drive transmission and similar others are well within the scope of the present invention. The power transmission mechanisms can act as speed increaser or speed reducer. In one embodiment, the extending rod (25) is defined with a connecting portion (25a) and an extending portion (25b). The extending portion (25b) is connected to the edge of the connecting portion (25a). The extending portion (25b) has an indent (25bi) that provides free to and fro movement of the object (10). The extending portion (25b) is defined with an extended edge (25bii). The extended edge has a hole (25biii) in which the second rotating arrangement (26) is introduced. The suspended rod (27) in connection with the second rotating arrangement (26) and suspends therefrom. More specifically, a coupler (26a) is used to couple the second rotating arrangement (26) with the suspended rod (27). The suspended rod (27) is defined with a free end (27a) which is provided with the object (10). If the object (10) is of a soft material, then the suspended rod (27) is directly injected into the object (10). If the object is a hard material, then the object (10) can be screwed or welded with the suspended rod (27). Alternatively, the suspended rod (27) and the object (10) can be connected by any other means, such as a locking arrangement, that establishes rigid connection there between. Yet in another embodiment, the object (10) can be an integral part of the suspended rod (27). In one embodiment, the extending rod (25) can be modified to have a telescopic arrangement for height adjustment.

In one embodiment, only the first rotating arrangement (24) is actuated that provides the revolutionary movement to the object (10) around the extending rod (25) to achieve a revolutionary movement of the eye or the eyes (12) of the user (11) by following the revolutionary movement of the object (10). Alternatively, only the second rotating arrangement (26) is actuated that provides the rotational movement of the object (10) to achieve a rotational movement of the eye or eyes (12) of the user (11) by following the rotational movement of the object (10). In another embodiment, both of the first rotating arrangement (24) and the second rotating arrangement (26) are actuated to achieve simultaneous revolutionary movement and rotational movement of the object (10). The simultaneous movement of the object (10) provides left to right, right to left, up to down, down to up, clockwise, counter clockwise, near and far movements of the eye or eyes (12) of the user (11). The simultaneous movement of the object (10) is the best operating mode as the eye or eyes (12) achieve the maximum continuous movements of left to right, right to left, up to down, down to up, clockwise, counter clockwise, near and far, thus rendering the most effective reduction of the eye(s) (12) strain.

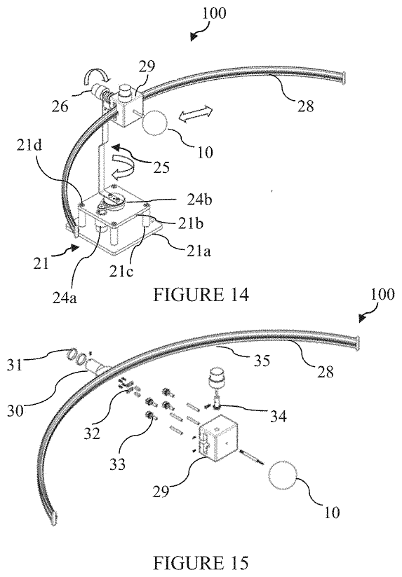

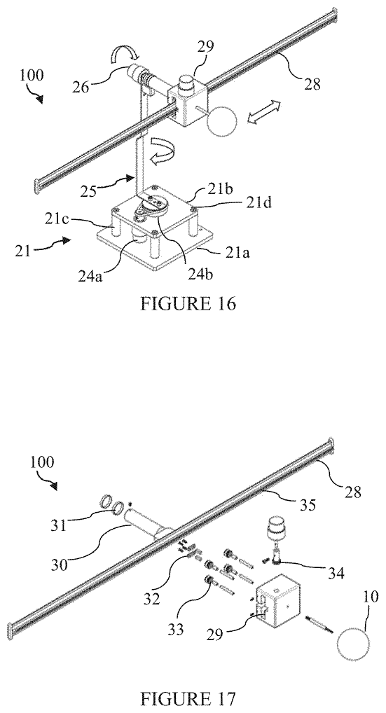

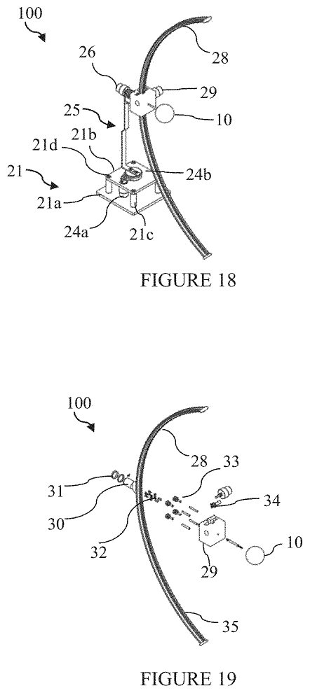

In an optional embodiment, the movement mechanism (20) further includes a track (28) and a slider arrangement (29). The track (28) is replaced with the suspended rod (27) and connected with the second rotating arrangement (26). In one embodiment, the track (28) has a connector (30) that is connected with the second rotating arrangement (26) on which two copper sleeves (31) are fitted. The connector (30) has two carbon brushes therewithin and a copper strip is mounted behind the track (28). A carbon brushed set (32) is disposed in the slider arrangement (29). The slider arrangement (29) includes a four V-shaped pulleys (33) that enables guided movement on the track (28) and a pinion gear (34) that engages with a rack (35) provided on the track (28). The track (28) can be curved (as illustrated in FIGS. 14-15 and 18-19) or flat (as illustrated in FIGS. 16-17 and 20-21). The track (28) is mountable horizontally (as illustrated in FIGS. 14 to 17) as well as vertically (as illustrated in FIG. 18-21). When the track (28) is horizontally mounted to achieve a horizontal configuration, then the slider arrangement (29) moves horizontally thereon and when the track (28) is vertically mounted to achieve a vertical configuration, then the slider arrangement (29) moves vertically thereon. The slider arrangement (29) is a slider motor and has the object (10) mounted thereon. The object (10) can be same object (10) disassembled from the suspended rod (27) or can be another object that can be identically same or can be different. During horizontal movement of the slider arrangement (29) and thereby the object (10), the eye or eyes (12) attains/achieves the horizontal movement. During vertical movement of the slider arrangement (29) and thereby the object (10), the eye or eyes (12) attains/achieves the vertical movement. The user (11) can re-arrange the configuration of the track (28).

In accordance with other embodiment, the movement mechanism (20) can only be provided with the configuration that provides only the rotational movement, the revolutionary movement and simultaneous rotational movement and revolutionary movement. In still another embodiment, the movement mechanism (20) can only be provided with the configuration that provides the horizontal movement, the vertical movement and the oscillating movement. In yet another embodiment, the movement mechanism (20) can be provided with a configuration that provides forward and reverse movement to the second rotating arrangement (26) so that the second rotating arrangement (26) can provide oscillating movement to the suspended rod (27). The second rotating arrangement (26) can be provided with a timer-based controller system that enables oscillation. Typically, the timer-based controller system (such as a Programmable Logic Controller (PLC) is well known in the art and the timer-based programming is easily programmed by a person skilled in the art) which includes an operating panel for providing the forward and reverse movement, a potentiometer and a recycle timer for setting the oscillation time. The programming of the timer-based controller can be altered as per the need of the movements of the object (10). The various configurations of the movement mechanism (20), as disclosed herein, can be selected based the need of the consumers and the commercial value at which the customer agrees to buy the product.

The present disclosure also includes a method for performing a user's (11) eye or eyes (12) exercise, in accordance with one embodiment of the present disclosure. The method is initiated by providing the eye exerciser (100) that has the object (10) and the movement mechanism (20). The movement mechanism (20) is configurable to direct controlled movement to the object (10) such that said object (10) attains at least one selected movement. The movement can be selected from the simultaneous rotating movement and revolutionary movement of object, the rotating movement, the revolutionary movement, the vertical movement, the horizontal movement and the oscillating movement. The provided eye exerciser (100) can either be positioned on the horizontal surface (22) (as illustrated in FIG. 1) or can be fixable on a vertical surface (23) (as illustrated in FIG. 4).

After providing and positioning the eye exerciser (100), the next step is actuating the movement mechanism (20) to attain the selected movement of the object (10) and the controlled movement of the object (10). The movement can be selected from the simultaneous rotating movement and revolutionary movement of object, the rotating movement, the revolutionary movement, the vertical movement, the horizontal movement and the oscillating movement. Herein, the rotating movement is rotation of the object (10) around the object's (10) rotational axis (X) and revolutionary movement is revolution of the object (10) around the defined-axis (Y) of the motion mechanism (20). In one embodiment, the method includes actuating the first rotating arrangement (24) such that the first rotating arrangement (24) rotates the extending rod (25) which is extending from the base arrangement (21). The base arrangement (21) supports the first rotating arrangement (24). The method further includes actuating the second rotating arrangement (26) such that the second rotating arrangement (26) rotates the suspended rod (27). The second rotating arrangement (26) is disposed at the extended edge (25bii) of the extending rod (25). The rotation of the suspended rod (27) provides rotational movement to the object (10) connected at the free end of the suspended rod (27). The rotation of the extending rod (25) results in revolutionary movement of the suspended rod (27) and thereby the object (10) around the defined-axis (X) of the extending rod (25).

In one embodiment, only the first rotating arrangement (24) is actuated that provides the revolutionary movement to the object (10) around the extending rod (25) to achieve a revolutionary movement of the eye or the eyes (12) of the user (11) by following the revolutionary movement of the object (10). Alternatively, only the second rotating arrangement (26) is actuated that provides the rotational movement of the object (10) to achieve a rotational movement of the eye or eyes (12) of the user (11) by following the rotational movement of the object (10). In the best working mode, both of the first rotating arrangement (24) and the second rotating arrangement (26) are actuated to achieve simultaneous revolutionary movement and rotational movement of the object (10).

The next step is following the controlled movement and the selected movement of the object (10) by the eye or eyes (12) of the user (12) who is at a distance from the object (10). The following of the eye or eyes (12) to the movement of the object (10) render eye or eyes (12) exercise. Further, the simultaneous movement of the object (10) provides left to right, right to left, up to down, down to up, clockwise, counter clockwise, near and far movements of the eye or eyes (12) of the user (11). The simultaneous movement of the object (10) is the best operating mode as the eye or eyes (12) achieve the maximum continuous movements of left to right, right to left, up to down, down to up, clockwise, counter clockwise, near and far, thus rendering the most effective reduction/relieving of the eye(s) (12) strain.

In an optional embodiment, the method includes performing the step of the vertical movement and the horizontal movement of the object (10) by:
a. dismantling the suspended rod (27) from the second rotating arrangement (26);
b. assembling the track (28) on the second rotating arrangement (26) selectively in the horizontal configuration and the vertical configuration;
c. mounting the slider arrangement (29) on the track (28), wherein the slider arrangement (29) is connected with the object (10); and
d. actuating the slider arrangement (29) such that the slider (29a) moves on the track (28);
   wherein, the movement of the slider (29a) is dependent on the position of the track (28) and following the movement of the slider (29a) render eye or eyes (12) exercise. The step of dismantling the suspended rod (27) and the assembling of the track (28) can be performed with or without tools. The tools used can be at least one of one or more screw drives and one or more spanners which are readily available in the market and hence not illustrated in the drawings.

In one embodiment, the first rotating arrangement (24), the second rotating arrangement (26) and the slider arrangement (29) can be manually operated. In another embodiment, the first rotating arrangement (24), the second rotating arrangement (26) and the slider arrangement (29) can be operated by the timer-based controller system such as the Programmable Logic Controller (PLC). The timer-based controller system provides oscillating movement to the suspended rod (27) to achieve the oscillating movement. The first rotating arrangement (24), the second rotating arrangement (26) and the slider arrangement (29) can be of a configuration that can provide rotation of the first rotating arrangement (24) and the second rotating arrangement (26) or the sliding movement of the slider arrangement (29) at a constant speed or at variable speeds. The selection of the first rotating arrangement (24), the second rotating arrangement (26) and the slider arrangement (29) thus can be made on the bases whether there is requirement of the constant speed or the variable speeds.

The foregoing description conveys the best understanding of the objectives and advantages of the present invention. Different embodiments, steps or alternatives may be made of the inventive concept of this invention. It is to be understood that all matter disclosed herein is to be interpreted merely as illustrative, and not in a limiting sense.

What is claimed is:

1. An eye exerciser comprising:
an object; and
a movement mechanism configurable to direct controlled movement to said object such that said object attain at least one selected movement, wherein, the selected movement of object is selected from the group consisting of:
a rotational movement;
a revolutionary movement;
a simultaneous rotational movement and a revolutionary movement;
a vertical movement;
a horizontal movement; and
an oscillating movement,
wherein, said rotating movement is rotation of said object around the object's rotational axis (X) and revolutionary movement is revolution of said object around the defined-axis (Y) of said motion mechanism, wherein, in an operative configuration, said movement mechanism configured to provide the selected and controlled movements to said object and a user moves an eye or eyes following the selected and controlled movements of said object rendering the eye or the eyes exercise.

2. The eye exerciser as claimed in claim 1, wherein said movement mechanism comprises:
a base arrangement positionable on a horizontal surface or fixable on a vertical surface;
a first rotating arrangement supported by said base arrangement;
an extending rod connected to said first rotating arrangement and extends from said base arrangement, said extending rod is controllably rotated by said first rotating arrangement;
a second rotating arrangement disposed at the extended edge of said extending rod; and
a suspended rod in connection with said second rotating arrangement and suspends therefrom, said suspended rod at free end provided with said object, wherein, in an operating configuration said first rotating arrangement rotates said suspended rod such that said object attain revolutionary movement around said extending rod and said second rotating arrangement rotates said suspended rod and said object to attain rotating movement, wherein the simultaneous revolutionary movement and the rotating movement render the eye or the eyes exercise.

3. The eye exerciser as claimed in claim 2, wherein said movement mechanism further includes:
a track replacing said suspended rod to be connected with said second rotating arrangement; and
a slider arrangement movable on said track, said slider arrangement connected with said object.

4. The eye exerciser as claimed in claim 3, wherein said track is connected with said second rotating arrangement in at least one of:
a vertical configuration to achieve the vertical movement; and
a horizontal configuration to achieve the horizontal movement.

5. The eye exerciser as claimed in claim 3, wherein said track is flat or curved.

6. A method for performing a user's eye or eyes exercise for relieving eye strain, said method comprising:
providing an eye exerciser having:
an object; and
a movement mechanism configurable to direct controlled movement to said object such that said object attain at least one selected movement,
actuating said movement mechanism to attain the selected movement of the object and the controlled movement of the object, the selected movement are one of:
a simultaneous rotating movement and revolutionary movement of said object;
a rotating movement;
a revolutionary movement;
a vertical movement;
a horizontal movement; and
an oscillating movement;
wherein, said rotating movement is rotation of said object around the object's rotational axis (X) and revolutionary movement is revolution of said object around the defined-axis (Y) of said motion mechanism; and
following the controlled movement and the selected movement of said object by the eye or eyes of the user who is at a distance from said object, render eye or eyes exercise.

7. The method as claimed in claim 6, wherein performing simultaneous revolutionary movement and rotational movement of said object by:
actuating a first rotating arrangement such that said first rotating arrangement rotates an extending rod extending from a base arrangement, said base arrangement supports said first rotating arrangement; and
actuating a second rotating arrangement such that said second rotating arrangement rotates a suspended rod, said second rotating arrangement disposed at the extended edge of said extending rod,
rotation of said suspended rod provides rotational movement to said object connected at the free end of said suspended rod,
wherein, the rotation of said extending rod results in revolutionary movement of said suspended rod and thereby the object around the defined-axis (X) of the extending rod,
following the simultaneous revolutionary movement and the rotating movement renders the eye or the eyes exercise.

8. The method as claimed in claim 7, wherein performing the step of the vertical movement and the horizontal movement of said object by:
dismantling said suspended rod from said second rotating arrangement;
assembling a track on said second rotating arrangement selectively in a horizontal configuration and a vertical configuration;
mounting a slider arrangement on said track, said slider arrangement connected with said object; and
actuating said slider arrangement such that a slider moves on said track;
wherein, the movement of said slider is dependent on the position of said track and following the movement of said slider render eye or eyes exercise.

9-10. (canceled)
